(19) United States
(12) Patent Application Publication
(10) Pub. No.: US 2002/0183335 A1
(43) Pub. Date: Dec. 5, 2002

(54) 2, 4-disubstituted pyrimidine-5-carboxamide derivatives as KCNQ potassium channel modulators

(76) Inventors: Piyasena Hewawasam, Middletown, CT (US); Dharmpal S. Dodd, Princeton, NJ (US); Charles D. Weaver, Wallingford, CT (US); Pierre Dextraze, Laprairie (CA); Valentin K. Gribkoff, Wallingford, CT (US); Gene G. Kinney, Collegeville, PA (US); Steven I. Dworetzky, Middlefield, CT (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/075,521

(22) Filed: Feb. 14, 2002

Related U.S. Application Data
(60) Provisional application No. 60/269,800, filed on Feb. 20, 2001.

(51) Int. Cl. A61K 31/505; A61K 31/513

(52) U.S. Cl. 514/256; 514/269

(57) ABSTRACT

There is provided a method of treatment for disorders responsive to the modulation of KCNQ potassium channels by administering to a mammal in need thereof a therapeutically effective amount of a 2,4-disubstituted pyrimidine-5-carboxamide derivative of the Formula I

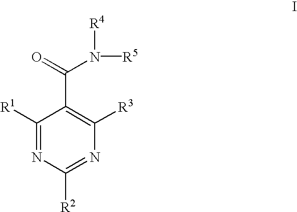

wherein R¹, R², R³, R⁴ and R⁵ are as defined below. The present invention also provides pharmaceutical compositions comprising openers or activators of the KCNQ potassium channels and especially to the method of treatment of disorders sensitive to KCNQ potassium channel opening activity such as migraine.

### CROSS-REFERENCE TO RELATED APPLICATIONS

This is a non-provisional application which claims the benefit of U.S. Provisional Application No. 60/269,800 filed Feb. 20, 2001.

### FIELD OF THE INVENTION

The present invention is directed to the use of 2,4-disubstituted pyrimidine-5-carboxamide derivatives which are modulators of KCNQ potassium channels and therefore are useful in treating disorders responsive to the modulation of the potassium channels. The present invention provides a method of treating disorders responsive to the modulation of the KCNQ potassium channels by administering to a mammal in need thereof a therapeutically effective amount of a 2,4-disubstituted pyrimidine 5-carboxamide derivative.

### BACKGROUND OF THE INVENTION

Potassium (K⁺) channels are considered to be the most diverse class of ion channels and have several critical roles in cell function. This has been demonstrated in neurons where K⁺ channels are responsible, in part, for determining cell excitability by contributing to membrane repolarization following depolarization, resting membrane potential, and regulation of neurotransmitter release. The M-current has long been described, by electrophysiology recording methods and by pharmacology, as a dominant conductance in controlling neuronal excitability. Pharmacological activation or suppression of M-currents by small molecules could have profound effects in controlling neuronal excitability. Recently, Wang et al. (1998, Science, 282:1890-1893) reported that co-assembly of the KCNQ2 and KCNQ3 potassium channels underlies one type of native M-current in neurons.

Activation or opening of the KCNQ channel(s), particularly the KCNQ2 or KCNQ3 channel(s), mutated or wild type, may prove to be beneficial in increasing hyperpolarization of neurons, thereby resulting in protection from abnormal synchronous firing during E a migraine attack. The present invention provides a solution to the problem of abnormal synchronous firing of neurons related to migraine headache by demonstrating that modulators, preferably openers, of KCNQ potassium channels increases hyperpolarization of neurons which protects against abnormal synchronous neuron firing involved in migraine attacks.

Although the symptom pattern varies among migraine sufferers, the severity of migraine pain justifies a need for vigorous, yet safe and effective, treatments and therapies for the great majority of cases. Needed in the art are agents that can be used to combat and relieve migraine (and diseases similar to and mechanistically related to migraine), and even prevent the recurrence of migraine. Also needed are anti-migraine agents which are effective in the treatment of acute migraine, as well as in the prodrome phase of a migraine attack. Thus, a clear goal in the art is to discover new, safe, nontoxic and effective anti-migraine compounds for use as drugs, and in anti-migraine compositions and treatments.

Because migraine afflicts a large percentage of the population, there is a need to discover compounds and agents that are useful in therapeutics and treatments, and as components of pharmaceutical compositions, for reducing, ameliorating, or alleviating the pain and discomfort of migraine headache and other symptoms of migraine. The present invention satisfies such a need by providing compounds that function as openers of the KCNQ family of potassium channel proteins to serve as anti-migraine agents or drugs and to comprise compositions to treat migraine, as described herein.

A number of substituted carboxyl-5-pyrimidine compounds have been disclosed in the art as neuroleptic agents by Bucker et a l. in U.S. Pat. No. 4,250,178 which issued on Feb. 10, 1981. A method for treating an inflammatory condition, such as immunoinflammatory and autoimmune diseases, by treating a warm-blooded animal in need thereof with pyrimidine carboxamide derivatives was disclosed by Suto et al. in U.S. Pat. No. 5,811,428 which issued on Sep. 22, 1998. Substitued pyrimidine carboxylates were also disclosed by Suto et al. in U.S. Pat. Nos. 5,852,028 issued Dec. 22, 1998 and 5,935,966 issued Aug. 10, 1999 as anti-inflammatory agents useful for the prevention and/or treatment of immunoinflammatory and autoimmune diseases. Thus, the compounds in the art and the uses described in these art patents are distinct from the novel use of the present invention.

### SUMMARY OF THE INVENTION

There is provided a method of treatment for disorders responsive to the modulation of KCNQ potassium channels by administering to a mammal in need thereof a therapeutically effective amount of a 2,4-disubstituted pyrimidine-5-carboxamide derivative of the Formula I

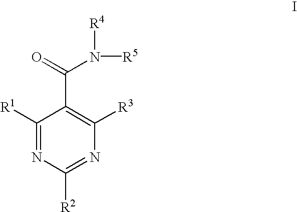

wherein R¹, R², R³, R⁴ and R⁵ are as defined below. The present invention also provides pharmaceutical compositions comprising openers or activators of the KCNQ potassium channels and to the method of treatment of disorders sensitive to KCNQ potassium channel opening activity such as migraine.

### DETAILED DESCRIPTION OF THE INVENTION

The present invention provides a method for the treatment or alleviation of disorders associated with KCNQ potassium channel polypeptides and, in particular, human KCNQ potassium channel polypeptides which are especially involved in reduction or alleviating migraine or a migraine attack, which comprises administering together with a conventional adjuvant, carrier or diluent a therapeutically effective amount of a compound of Formula I

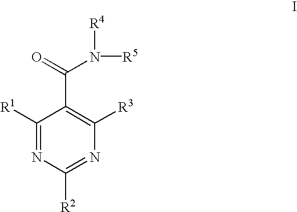

wherein

R¹ is selected from hydrogen, halogen, C₁₋₈alkyl, phenyl, phenylalkyl, C₃₋₆heterocyclic, C₃₋₆heterocyclicmethyl, —CN, —OR, —NRR, —NRNCOR or —CF₃;

R² is selected from halogen, C₁₋₈alkyl, C₃₋₇cycloalkyl, phenyl, phenylalkyl, C₃₋₆heterocyclic, C₃₋₆heterocyclicmethyl, —CN, —OR, —NRR, —NRNCOR or —S—R;

R³ is selected from hydrogen, halogen or C₁₋₈alkyl;

R⁴ is selected from hydrogen, —CH₃ or —CH₂C₆H₅;

R⁵ is selected from hydrogen, C₁₋₈alkyl, C₃₋₇cycloalkyl, phenyl, phenylalkyl, C₃₋₆heterocyclic or C₃₋₆heterocyclicmethyl;

and wherein each occurrence of R is independently selected from the groups consisting of C₁₋₈alkyl, C₃₋₇alkynyl, phenyl, phenylalkyl, C³⁻⁶heterocyclic and C₃₋₆heterocyclicmethyl.

The terms “C₁₋₄ alkyl” and “C₁₋₈ alkyl” as used herein and in the claims means a straight or branched chain alkyl group containing from 1 to 8 carbon atoms such as methyl, ethyl, propyl, isopropyl, butyl, sec-butyl, isobutyl, tert-butyl, pentyl, isopentyl, amyl, hexyl, isohexyl and the like. Preferably, these groups contain from 1 to 4 carbon atoms. The term “C₃₋₇ cycloalkyl” means a carbon cyclic ring system such as cyclopropyl, cyclobutyl, cyclopentyl, cyclohexyl and cycloheptyl. The term “C₃₋₇ alkynyl” means a straight or branched chain alkynyl group containing 3 to 7 carbon atoms such as 2-propyn-1-yl, 4-pentyn-1-yl, 2-butyn-1-yl, 2-methyl-3-butyn-2-yl, 3-butyn-2-yl and the like. The term “halogen” is intended to include bromo, chloro, iodo, and fluoro. The term “phenylalkyl” means a straight or branched chain C₁₋₄ alkyl group containing an aromatic phenyl moiety such as phenylmethyl, phenylethyl, phenylbutyl and the like. The term “C₃₋₆ heterocyclic” means a heterocyclic ring system containing from 3 to 6 carbon atoms and one or more hetero atoms such as pyrrolyl, furanyl, thienyl, imidazolyl, oxazolyl, thiazolyl, pyrazolyl, pyrrolidinyl, pyridinyl, pyrimidinyl, purinyl and the like.

In the method of the present invention, the term “therapeutically effective amount” means the total amount of each active component of the method that is sufficient to show a meaningful patient benefit, i.e.; amelioration or healing of conditions which respond to modulation of the KCNQ potassium channels. When applied to an individual active ingredient, administered alone, the term refers to that ingredient alone. When applied to a combination, the term refers to combined amounts of the active ingredients that result in the therapeutic effect, whether administered in combination, serially or simultaneously. The term “KCNQ” as used herein and in the claims means the family of KCNQ2, KCNQ3, KCNQ4, and KCNQ5 potassium channel polypeptides as well as heteromultimers of different individual family members which include but are not limited to KCNQ2/3, KCNQ2/5 and KCNQ3/5. The terms “treat, treating, treatment” as used herein and in the claims means preventing, alleviating or ameliorating diseases and/or symptoms associated with dysfunction of cellular membrane polarization and conductance of human KCNQ2, KCNQ3, KCNQ4, and KCNQ5 potassium channel polypeptides and, in particular, migraine and/or symptoms that precede a full-blown migraine attack.

The present invention provides a method of treatment for disorders responsive to the modulation of KCNQ potassium channels by administering to a mammal in need thereof a therapeutically effective amount of a 2,4-disubstituted pyrimidine-5-carboxamide derivative. Methods for preparing 2,4-disubstituted pyrimidine-5-carboxamide derivatives have been disclosed by Suto et al. in U.S. Pat. No. 5,811,428 which issued on Sep. 22, 1998.

The general procedures used to synthesize the compounds of Formula I are described in Reaction Schemes 1-7 and are illustrated in the examples. Reasonable variations of the described procedures, which would be evident to one skilled in the art, are intended to be within the scope of the present invention.

2-Aminopyrimidine-5-carboxamide derivatives of Formula Ia can be prepared from corresponding β-keto esters of Formula II by following the general procedure shown below in Scheme 1.

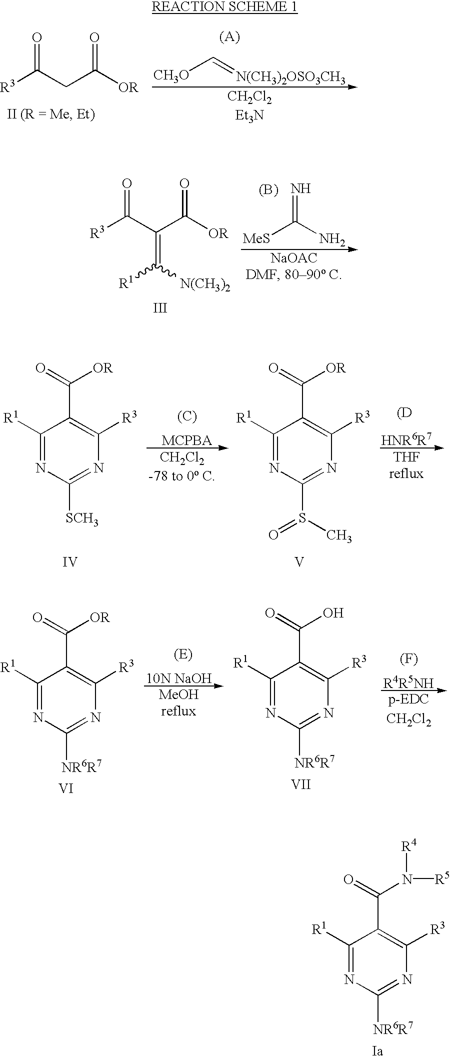

Step A in Reaction Scheme 1 depicts the preparation of the enaminone intermediate of Formula III wherein R is methyl. The procedure employed for the preparation of intermediates of Formula III may be described by the following preparation. To a solution of an appropriate methyl-3-oxopropionate intermediate of Formula II (R=methyl; 18.8 mmol) and dimethylformamide-dimet h iyl sulfate adduct (5.0 g, 25.1 mmol, prepared by reacting a mixture of 1.05 mol of dimethylformamide and 1.0 mol of Me₂SO₄ at 40° C. for 4 hours and at room temperature for 48 hours) in dichloromethane (30 mL), was added triethylamine (3.8 mL, 27.3 mmol) at 0° C. The reaction mixture was stirred at room temperature for 16 hours and then washed consecutively with 10% tartaric acid and water and then the organic layer was dried over MgSO₄, filtered, and the filtrate concentrated in vacuo. The isolated crude material was purified by flash column chromatography (silica gel eluted with 1:2 hexanes:ethyl acetate) to afford an intermediate enaminone of Formula III (R=methyl).

Step B in Reaction Scheme 1 shows the preparation of a 2-(methylthio)pyrimidine-5-carboxylic acid, methyl ester intermediate of Formula IV wherein R is methyl. The procedure employed for the preparation of intermediates of Formula IV may be described by the following preparation. To a mixture of 2-methylisothiourea sulfate (1.85 g, 6.66 mmol) and sodium acetate (2.28 g, 27.75 mmol) in dimethylformamide (20 mL), was added the enaminone intermediate of Formula III (R=methyl, 11.1 mmol). The reaction mixture was heated at 80-90° C. for 16 hours. The reaction was cooled to room temperature and then diluted with water. Precipitated off-white solid was collected to afford the intermediate of Formula IV.

Step C in Reaction Scheme 1 depicts the preparation of a 2-(methylsulfinyl)pyrimidine-5-carboxylic acid, methyl ester intermediate of Formula V wherein R is methyl. The procedure employed for the preparation of the intermediates of Formula V may be described by the following preparation. To a solution of intermediate of Formula IV (5.63 mmol) in dichloromethane (30 mL), was added 3-chloroperoxybenzoic acid (1.17 g, 6.76 mmol) at −78° C. The reaction mixture was stirred in an ice bath for 3 hours. The reaction mixture was washed consecutively with saturated sodium bicarbonate solution and brine. The organic layer was dried over MgSO₄, filtered, and the filtrate was concentrated in vacuo to provide the intermediate of Formula V.

Step D in Reaction Scheme 1 depicts the preparation of a 2-(substituted amino)pyrimidine-5-carboxylic acid, methyl ester intermediate of Formula VI wherein R is methyl. The procedure employed for the preparation of the intermediates of Formula VI may be described by the following preparation. To a solution of intermediate of Formula V (1.41 mmol) in THF (3 mL), was added an appropriate primary or secondary amine of formula HNR⁶R⁷ (2.83 mmol). The reaction mixture was heated under reflux for 3 hours. The solvent and excess amine were removed in vacuo. The resultant residue was washed with saturated sodium bicarbonate solution to give the intermediate of Formula VI.

Step E in Reaction Scheme 1 shows the preparation of a 2-(substituted amino)pyrimidine-5-carboxylic acid intermediate of Formula VII. The procedure employed for the preparation of the intermediates of Formula VII may be described by the following preparation. A solution of intermediate of Formula VI (0.68 mmol) in 10 N sodium hydroxide (5 mL) and methanol (5 mL) was heated under reflux for 3 hours. The solvents were removed in vacuo. The resultant aqueous residue was neutralized with 1 N HCl to pH 7. The carboxylic acid intermediate of Formula VII was then collected as a solid.

Step F in Reaction Scheme 1 depicts the preparation of the 2-(substituted amino)pyrimidine-5-carboxamide compound of Formula Ia. The compound of Formula Ia may be prepared as follows: To a solution of intermediate VII (0.08 mmol) in dichloromethane (2 mL), polymer supported 1-(3-dimethylaminopropyl)-3-ethylcarboc iimide resin (457 mg, 0.64 mmol) and an appropriate primary or secondary amine of formula HNR⁴R⁵ (0.16 mmol) were added. The reaction mixture was stirred at room temperature for 16 hours. The resin was filtered off and the solvent was removed in vacuo. The resultant residue was purified by preparative HPLC to afford the compound of Formula Ia, isolated as the TFA salt. The compounds of Examples 1 through 18 were prepared by following the general procedures of steps A through F as described above for Reaction Scheme 1.

The 2-(substituted amino)pyrimidine-5-carboxamide derivatives of Formula Ia may also be prepared from a 2-chloropyrimidine-5-carbonyl chloride of Formula VIII by following the general procedures described in Reaction Scheme 2.

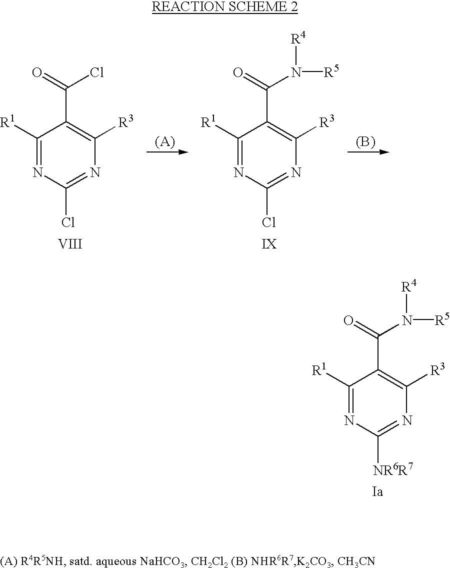

Step A of Reaction Scheme 2 shows the preparation of intermediates of Formula IX by treating a solution of a 2-chloropyrimidine-5-carbonyl chloride of Formula VIII (3 mmol) in dichloromethane (5 mL) with saturated sodium bicarbonate (5 mL) and an appropriate primary or secondary amine of formula R⁴R⁵NH (3.3 mmol). The reaction mixture was stirred at room temperature for 3 hours. The precipitated solid intermediate of Formula IX was collected by filtration and then dissolved in acetonitrile (10-15 mL). To the resulting solution was added potassium carbonate (0.62 g, 4.5 mmol) and an appropriate amine of formula HNR⁶R⁷ (6 mmol). The reaction mixture was stirred at room temperature overnight. The inorganic salts were filtered off and the filtrate was concentrated to afford the compound of Formula Ia. The compounds of Examples 19 through 30 were prepared by following the general procedure described above in Reaction Scheme 2.

Reaction Scheme 3 shows the conversion of a 2,4-dihydroxypyrimidine-5-carboxylic acid of Formula X to a 2-(substituted amino)pyrimidine-5-carboxamide derivative of Formula Ia.

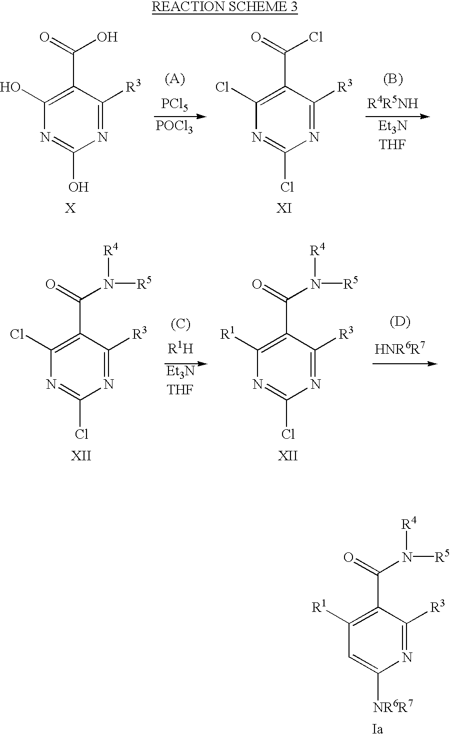

Step A in Reaction Scheme 3 shows the conversion of a 2,4-dihydroxypyrimidine-5-carboxylic acid to a 2,4-dichloropyrimidine-5-carbonyl chloride which can be carried out by refluxing a suspension of a 2,4-dihydroxypyrimidine-5-carboxylic acid of Formula X (0.128 mol) in POCl₃ (700 mL) for 11 hours. The reaction mixture was then cooled to 23° C., treated with PCl₅, and then refluxed for an additional 16 hours. The reaction mixture was then cooled to 23° C. and concentrated under reduced pressure to give a thick syrup. Traces of volatile phosphorus derivatives were co-distilled twice with toluene (2×250 mL) leaving intermediate of Formula XI as a thick syrup [see also Stogryn, E. L. J. Med. Chem., 1972, 15(2), 200-201]. The crude intermediate of Formula XI is then used without further purification in Step B.

Step B in Reaction Scheme 3 shows the preparation of a 2,4-dichloropyrimidine-5-carboxamide derivative of Formula XII. The crude 2,4-dichloropyrimidine-5-carbonyl chloride derivative of Formula XI (0.047 mol) was diluted in dry THF (200 mL), the solution was cooled to −78° C., then Et₃N was added in one portion (20 mL, 0.142 mol). The cold mixture was treated dropwise with one equivalent of an appropriate amine of the formula R⁴R⁵NH (0.047 mol), stirred for 1 hour, diluted with HCl (0.5 N, 200 mL) and then extracted with ethyl acetate. The organic layer was dried over MgSO₄, filtered and the filtrate was concentrated in vacuo to afford the crude 2,4-dichloropyrimidine-5-carboxamide derivative of Formula XII. Re-crystallization of the crude product of Formula XII from a solvent mixture such as THF-hexanes afforded a pure 2,4-dichloropyrimidine-5-carboxamide derivative of Formula XII.

Step C in Reaction Scheme 3 shows the preparation of a 2-chloro-4-(substituted amino)pyrimidine-5-carboxamide derivative from the corresponding 2,4-dichloropyrimidine-5-carboxamide derivative. The 2,4-dichloropyrimidine-5-carboxamide derivative is reacted with an appropriate amine of the formula R¹H (wherein R¹ represents the disubstituted nitrogen of the amine) in an appropriate solvent, such as THF, in the presence of a base, such as triethylamine, to afford after workup the intermediate of Formula IX.

The intermediate of Formula IX may then be reacted with an appropriate amine of the formula HNR⁶R⁷ to afford the compound of Formula Ia. When HNR⁶R⁷ is NH₃ the following procedure may be employed. A solution of the intermediate of Formula IX (1.56 mmol) in 1-methyl-2-pyrrolidinone (25 mL) was cooled to 0° C. and saturated with NH₃ in a steel bomb. The steel bomb was sealed and heated at 120° C. for 24 hours. After cooling to 23° C., the mixture was diluted with water and extracted with ethyl acetate. The combined organic extracts were washed with water, dried over MgSO₄, filtered and the filtrate was concentrated in vacuo. The residue was triturated with diethyl ether and collected by filtration to afford a compound of Formula Ia (wherein R⁶ and R⁷ are hydrogen) as a solid. Alternatively, compounds of Formula Ia may be prepared by reacting intermediate of Formula IX with amines of formula HNR⁶R⁷ (wherein R⁶ and R⁷ are not both hydrogen) according to the following method. To a solution of intermediate of Formula IX (0.025 mmol) in 1-methyl-2-pyrrolidinone (0.5 mL) was added a 1.0 M solution of amine derivative of formula HNR⁶R⁷ (0.125 ml, 0.125 mmol, 5 eq.) in 1-methyl-2-pyrrolidinone. The resulting mixture was heated for 18 hours at a temperature of 100° C. to 135° C. The crude mixture was purified by HPLC (PRIMESPHERE C18, 21.1 mm×100 mm column); mobile phase: A 10/90 CH₃CN/H₂0+5 mMol NH₄OAc, B 90/10 CH₃CN/H₂0+5 mMol NH₄OAc; gradient: 40% to 0% of A over 5 minutes; detector: UV, 220 nM; Flow rate: 20.0 ml/min. Purity of samples were analyzed by LCMS: HPLC (LUNA C8, 5μ, 4.6 mm×30 mm column); mobile phase: A 10/90 CH₃CN/H₂0+5 mMol NH₄OAc, B 90/10 CH₃CN/H₂0+5 mMol NH₄OAc; gradient: 100% to 0% of A over 4 minutes; detector: UV, 250 nM; Flow rate: 4.0 mL/min; or (Primesphere C18-HC, 4.6 mm×30 mm column); mobile phase: A 10/90 CH₃CN/H₂0+5 mMol NH₄OAc, B 90/10 CH₃CN/H₂0+5 mMol NH₄OAc; gradient: 100% to 0% of A over 3 minutes; detector: UV, 250 nM; Flow rate: 4.0 mL/min.

The 2-aryloxypyrimidine-5-carboxamide derivatives of Formula Ib may be prepared by reacting the intermediate of Formula IX with the potassium salt of an appropriate phenol derivative as shown in Reaction Scheme 4.

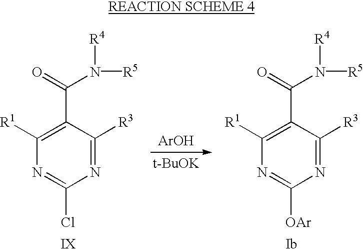

To a solution of intermediate of Formula IX (0.025 mmol) in 1-methyl-2-pyrrolidinone (0.5 mL) was added successively a 1.0 M solution of an appropriate phenol derivative of formula ArOH (0.125 mL, 0.125 mmol, 5 eq.) in 1-methyl-2-pyrrolidinone and a solution of potassium tert-butoxide in THF (1.0 M, 0.125 mL, 0.125 mmol, 5 eq.). The resulting mixture was heated at 85° C. for 15 hours. After cooling to 23° C., the mixture was quenched by addition of an aqueous solution of NaH₂PO₄ (1.0 M, 0.25 ml) and filtered on PTFE filter prior to purification by HPLC (PRIMESPHERE C18-HC, 21.2 mm×100 mm column); mobile phase: A is 10/90 CH₃CN/H₂0+5 mMol NH₄OAc, B is 90/10 CH₃CN/H₂0+5 mMol NH₄OAc; gradient: 40% to 0% of A over 5 minutes; detector: UV, 220 nM; Flow rate: 20.0 mL/min. Purity of each sample was analyzed by LCMS: HPLC (Primesphere C18-HC, 4.6 mm×30 mm column); mobile phase: A is 10/90 CH₃CN/H₂0+5 mMol NH₄OAc, B is 90/10 CH₃CN/H₂0+5 mMol NH₄OAc; gradient: 100% to 0% of A over 3 minutes; detector: UV, 250 nM; Flow rate: 4.0 mL/min.

The 2-alkoxypyrimidine-5-carboxamide derivatives of Formula Ic may be prepared by reacting intermediate of Formula IX with the sodium salt of an appropriate alcohol derivative, ROH, as shown in Reaction Scheme 5.

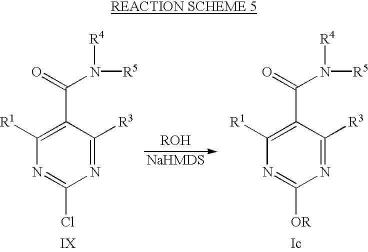

To a solution of intermediate of Formula IX (0.050 mmol) in dioxane (0.6 mL) was added successively a 1.0 M solution of the desired alcohol (0.40 mL, 0.40 mmol, 8 eq.) in dioxane and a 1.0 M solution of sodium hexamethyldisilazide (NaHMDS) in THF (0.250 mL, 0.250 mmol, 5 eq.). The resulting mixture was heated at 70° C. for 2 hours. After cooling at 23° C., the mixture was quenched by addition of an aqueous solution of NH₄Cl (1.0 N, 0.40 ml) and filtered on PTFE filter. The sticky material was removed from vials by addition of MeOH and the resulting solution was filtered. The crude filtrates were combined and purified by HPLC (PRIMESPHERE C18-HC, 21.2 mm×100 mm); mobile phase: A 10/90 CH₃CN/H₂0+5 mMol NH₄OAc, B 90/10 CH₃CN/H₂0+5 mMol NH₄OAc; gradient: 40% to 0% of A over 5 minutes; detector: UV, 220 nM; Flow rate: 20.0 ml/min. Purity of each sample was analyzed by LCMS: HPLC (YMC ODS-A C18, 4.6 mm×33 mm); mobile phase: A 10/90 CH₃CN/H₂0+5 mMol NH₄OAc, B 90/10 CH₃CN/H₂0+5 mMol NH₄OAc; gradient: 100% to 0% of A over 3 minutes; detector: UV, 220 nM; Flow rate: 4.0 mL/min.

2-Arylpyrimidine-5-carboxamide derivatives of Formula Id may be prepared by Pd(0) mediated coupling of an intermediate of Formula IX with an appropriate aryl boronic acid derivative, ArB(OH)2 as depicted in Reaction Scheme 6 and by the following procedure.

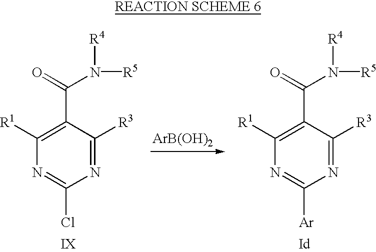

To a solution of intermediate IX (0.052 mmol) in 1-methyl-2-pyrrolidinone (1.0 mL) was added an appropriate boronic acid derivative, ArB(OH)₂ (1.17 mmol, 2.25 eq.) and an aqueous solution of NaHCO₃ (2.0 M, 0.10 mL). The resulting mixture was flushed with argon prior to the addition of tetrakis triphenylphosphine palladium [Pd(PPh₃)₄, (0.003 g)] and then heated at 110° C. for 2 hours. After cooling to 23° C., the mixture was purified by HPLC (PRIMESPHERE C18-HC, 21.2 mm×100 mm column); mobile phase: A is 10/90 CH₃CN/H₂0+5 mMol NH₄OAc, B is 90/10 CH₃CN/H₂0+5 mMol NH₄OAc; gradient: 70% to 0% of A over 8 minutes; detector: UV, 220 nM; Flow rate: 20.0 mL/min. Purity of each sample was analyzed by LCMS: HPLC (Primespher C 18-HC, 4.6 mm×30 mm column); mobile phase: A is 10/90 CH₃CN/H₂0+5 mMol NH₄OAc, B is 90/10 CH₃CN/H₂0+5 mMol NH₄OAc; gradient: 100% to 0% of A over 3 minutes; detector: UV, 250 nM; Flow rate: 4.0 mL/min.

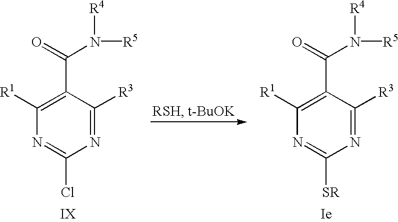

Reaction Scheme 7 depicts the reaction of an intermediate of Formula IX with an appropriate thiol in the presence of an appropriate base such as potassium tert-butoxide to provide compounds of Formula Ie. The reaction may be carried out by the following preparation. To a solution of intermediate of Formula IX (0.025 mmol) in 1-methyl-2-pyrrolidinone (0.5 mL) was added a 1.0 M solution of thiol derivative, RSH (0.125 mL, 0.125 mmol, 5 eq.) in 1-methyl-2-pyrrolidinone and a solution of potassium tert-butoxide in THF (1.0 M, 0.125 mL, 0.125 mmol, 5 eq.).

The resulting mixture was heated at 80° C. for 2 hours. After cooling at 23° C., the mixture was quenched by the addition of aqueous solution of NH₄Cl (1.0 M, 0.3 mL) and purified by HPLC (PRIMESPHERE C18-HC, 21.2 mm×100 mm column); mobile phase: A is 10/90 CH₃CN/H₂0+5 mMol NH₄OAc, B is 90/10 CH₃CN/H₂0+5 mMol NH₄OAc; gradient: 40% to 0% of A over 5 minutes; detector: UV, 220 nM; Flow rate: 20.0 mL/min. Purity of each sample was analyzed by LCMS: HPLC (Primesphere C18-HC, 4.6 mm×30 mm column); mobile phase: A is 10/90 CH₃CN/H₂0+5 mMol NH₄OAc, B is 90/10 CH₃CN/H₂0+5 mMol NH₄OAc; gradient: 100% to 0% of A over 3 minutes; detector: UV, 250 nM; Flow rate: 4.0 mL/min.

### BIOLOGICAL ACTIVITY

KCNQ Oocyte Methods and Results

Potassium (K⁺) channels are structurally and functionally diverse families of K⁺-selective channel proteins which are ubiquitous in cells, indicating their central importance in regulating a number of key cell functions [Rudy, B., Neuroscience, 25: 729-749 (1988)]. While widely distributed as a class, K⁺ channels are differentially distributed as individual members of this class or as families. [Gehlert et al., Neuroscience, 52: 191-205 (1993)]. In general, activation of K⁺ channels in cells, and particularly in excitable cells such as neurons and muscle cells, leads to hyperpolarization of the cell membrane, or in the case of depolarized cells, to repolarization. In addition to acting as an endogenous membrane voltage clamp, K⁺ channels can respond to important cellular events such as changes in the intracellular concentration of ATP or the intracellular concentration of calcium (Ca²⁺). The central role of K⁺ channels in regulating numerous cell functions makes them particularly important targets for therapeutic development. [Cook, N. S., Potassium channels: Structure, classification, function and therapeutic potential. Ellis Horwood, Chinchester (1990)]. One class of K+ channels, the KCNQ family exemplified by KCNQ2, KCNQ2/3 heteromultimeres, and KCNQ5, is regulated by transmembrane voltage and plays a potentially important role in the regulation of neuronal excitability [Biervert et al., Science, 279: 403-406 (1998); Lerche et al., J. Biol. Chem. 275:22395-22400 (2000); Wang et al., Science, 282:1890-1893 (1998)].

An opener of KCNQ channels, such as the KCNQ2 and KCNQ2/3 channel opener retigabine, exerts its cellular effects by increasing the open probability of these channels [Main J., Mol Pharmacol 58(2):253-62 (2000); Wickenden et al.,. Mol. Pharm. 58:591-600 (2000)]. This increase in the opening of individual KCNQ channels collectively results in the hyperpolarization of cell membranes, particularly in depolarized cells, produced by significant increases in whole-cell KCNQ-mediated conductance.

The ability of compounds described in the present invention to open KCNQ channels and increase whole-cell outward (K⁺) KCNQ-mediated currents was assessed under voltage-clamp conditions by determining their ability to increase cloned mouse KCNQ2 (mKCNQ2)-mediated, heteromultimeric KCNQ2/3 (KCNQ2/3)-mediated, and human KCNQ5 (hKCNQ5)-mediated outward currents heterologously expressed in Xenopus oocytes. Oocytes were prepared and injected using standard techniques; each oocyte was injected with approximately 50 nl of mKCNQ2, or hKCNQ5 cRNA. In the case of mKCNQ2/3 heteromultimeric channel expression, equal amounts (25-50 nL) of each cRNA were co-injected. Injection of equivalent amounts of water (50 nl) did not result in expression of outward currents at the voltage steps used to detect KCNQ expression. Following injection, oocytes were maintained at 17° C. in ND96 medium consisting of (in mM): NaCl, 90; KCl, 1.0; CaCl₂, 1.0; MgCl₂, 1.0; HEPES, 5.0; pH 7.5. Horse serum (5%) and penicillin/streptomycin (5%) were added to the incubation medium. Recording commenced 2-6 days following mRNA injection. Prior to the start of an experiment oocytes were placed in a recording chamber and incubated in Modified Barth's Solution (MBS) consisting of (in mM): NaCl, 88; NaHCO₃, 2.4; KCl, 1.0; HEPES, 10; MgSO₄, 0.82; Ca(NO₃)₂, 0.33; CaCl₂, 0.41; pH 7.5.

Oocytes were impaled with electrodes (1-2 MΩ) and standard 2-electrode voltage clamp techniques were employed to record whole-cell membrane currents. Recordings were accomplished using standard two-electrode voltage clamp techniques [Stuhmer et al., Methods in Enzymology, Vol. 207: 319-339 (1992)]. Voltage-clamp protocols typically consisted of a series of voltage steps 1-5 sec duration, in +10 mV steps from a holding potential of −90 mV to a maximal potential of +40 mV; records were digitized at 5 kHz and stored on a computer using pclamp data acquisition and analysis software (Axon Instruments). Compounds were evaluated at a single concentration (10 or 20 μM); the effect of the selected compounds of Formula I on KCNQ2 current was expressed as the percent of control current and is listed in Table I.

TABLE 1
Effect of Selected Compounds on KCNQ2 Channels

| | KCNQ2 |
|---|---|
| Example No. | Current* |
| 1 | +++ |
| 4 | +++ |
| 9 | +++ |
| 19 | ++** |
| 37 | ++ |
| 39 | + |

*Unless otherwise noted, at 20 μM expressed as percent increase over KCNQ current in controls;
**at 5 μM
+ = 125-150%
++ = 151-200%
+++ = >200%

In Vivo Electrophysiology

Male Long-Evans rats (Harlan, 250-400 g) were used in the experiments described in this example. Prior to testing, rats were allowed access to food and water ad libitum and were maintained on a 12:12-h light/dark cycle. Rats were group housed in an Association for Assessment and Accreditation of Laboratory Animal Care (AAALAC) accredited facility and cared for in strict compliance with all applicable regulations.

Superior sagital sinus (SSS) stimulation and recording were performed in a manner consistent with previously published methods using cat (Hoskin et al., 1996) and rat (Cumberbatch et al, 1998; 1999) animal models. Rats were anesthetized with 1.2 g/kg i.p. urethane (#U-2500, Sigma Chemical Company, St. Louis, Mo.) and given supplemental urethane as needed. In the case of intravenous (i.v.) drug administration, the jugular veins of the rats were cannulated using sylastic tubing pre-filled with vehicle.

Rats were placed in a stereotaxic device (#1730, David Kopf Instruments, Tujunga, Calif.) and an incision was made to expose the entire skull that continued caudally to the level of the C1/C2 vertebral juncture. Using a microdrill (#770, Dremel, Racine, Wis.) and #4 carbide burr (Henry Schein, Melville, N.Y.), a square section of skull was removed extending from the bregma position, rostrally, to the lambda position, caudally. The underlying dura mater was incised bilateral to the SSS and a small section of Parafilm® (American National Can, Neenah, Wis.) was placed under the SSS to isolate the stimulation electrode. The SSS was stimulated using insulated silver electrodes bent at their ends to form a hook. The dorsal region of the vertebra corresponding to C2 was removed for access to the trigeminal nucleus caudalis.

Stimulated field responses were recorded in the trigeminal nucleus caudalis using Teflon coated stainless-steel microelectrodes (5 megaohms impedance, Frederick Haer, Brunswick, Me.) and amplified and filtered (0.1 Hz-10 kHz) using a differential amplifier (#IsoDAM8, World Precision Instruments, Sarasota, Fla.). Stimulation voltage (250 μsec, 40-130V) was delivered using a Grass S88 (Grass Medical Instruments, Quincy, Mass.) stimulator and stimulus isolation unit (Grass #SIU5) at a rate of 0.3 Hz. Amplified potentials were captured with an analog-to-digital converter (#1401 plus, Cambridge Electronic Design, Cambridge, UK) and commercially available software (#Signal, Cambridge Electronic Design). Low temperature wax was applied to both the recording and stimulation sites to prevent dehydration.

Three baseline measures (i.e., 100% of control), each consisting of 100 evoked trigeminal field potentials, were sampled prior to drug injection. The primary measure for efficacy were changes in trigeminal field potential amplitude following injection of test compound. A decrease in trigeminal field response amplitude was considered to evidence anti-migraine activity. Following injection of test substances, data were sampled for 1 hour, averaged into 5 minute bins (90 evoked potentials) and expressed as a percent change from average baseline values for the purposes of statistical analysis. Data were analyzed using repeated measures analyses of variance comparing vehicle and drug effects. A difference was considered significant when p<0.05.

In one embodiment of the present invention, openers or activators of the KCNQ2 potassium channel protein have been found to be effective in the above-described model of migraine involving vasculo-trigeminal systems which are integrally involved in the transmission of migraine pain. A non-limiting representative compound used in the SSS-stimulated trigeminal model for migraine as described in Example 19 produced a dose-dependent reduction in the SSS-stimulated trigeminal field response (overall ANOVA, p<0.001). The compound of Example 19 was prepared as a solution in 100% polyethylene glycol (MW=400) using sonication to aid in dissolution and administered via the i.v. catheter described above at a maximum volume of 0.3 cc. At a dose of 1 mg/kg i.v., the compound of Example 19 produced a statistically significant 25.2% (p=0.005) decrease in field potential compared with vehicle at 60 minutes following i.v. injection in the superior sagittal sinus (SSS) model of migraine.

The results of the KCNQ potassium channel openers described above demonstrate that the compounds of the present invention results in the hyperpolarization of cell membranes and for the in vivo SSS-field potential experiments demonstrate that the KCNQ2 openers are useful for modulating neuronal activity and may result in protection from abnormal synchronous firing during a migraine attack. Accordingly, the KCNQ opener or activator compounds described according to the present invention are capable of limiting neuronal activity within the trigeminovascular system and are thus particularly useful for the treatment of migraine headache and migraine attack in individuals suffering from the pain and discomfort of migraine. The compounds of the present invention are therefore useful in the treatment of acute migraine, as well as the potential for prophylactic treatment of migraine as demonstrated by efficacy in a model of cortical spreading depression. Furthermore, the compounds of the present invention could reduce, ameliorate, eliminate or prevent one, or a number of, the characteristic cluster of symptoms, namely, nausea, photophobia, phonophobia and basic functional disabilities, that are further associated with migraine and migraine pain that occur after the prodrome phase of a migraine headache.

In another embodiment, this invention relates to a method of treatment or prevention of disorders responsive to opening of KCNQ potassium channels in a mammal in need thereof, which comprises administering to said mammal a therapeutically effective amount of a compound of Formula I.

For therapeutic use, the pharmacologically active compounds of Formula I will normally be administered as a pharmaceutical composition comprising as the (or an) essential active ingredient at least one such compound in association with a solid or liquid pharmaceutically acceptable carrier and, optionally, with pharmaceutically acceptable adjutants and excipients employing standard and conventional techniques.

The pharmaceutical compositions include suitable dosage forms for oral, parenteral (including subcutaneous, intramuscular, intradermal and intravenous) bronchial or nasal administration. Thus, if a solid carrier is used, the preparation may be tableted, placed in a hard gelatin capsule in powder or pellet form, or in the form of a troche or lozenge. The solid carrier may contain conventional excipients such as binding agents, fillers, tableting lubricants, disintegrants, wetting agents and the like. The tablet may, if desired, be film coated by conventional techniques. If a liquid carrier is employed, the preparation may be in the form of a syrup, emulsion, soft gelatin capsule, sterile vehicle for injection, an aqueous or non-aqueous liquid suspension, or may be a dry product for reconstitution with water or other suitable vehicle before use. Liquid preparations may contain conventional additives such as suspending agents, emulsifying agents, wetting agents, non-aqueous vehicle (including edible oils), preservatives, as well as flavoring and/or coloring agents. For parenteral administration, a vehicle normally will comprise sterile water, at least in large part, although saline solutions, glucose solutions and like may be utilized. Injectable suspensions also may be used, in which case conventional suspending agents may be employed. Conventional preservatives, buffering agents and the like also may be added to the parenteral dosage forms. Particularly useful is the administration of a compound of Formula I directly in parenteral formulations. The pharmaceutical compositions are prepared by conventional techniques appropriate to the desired preparation containing appropriate amounts of the active ingredient, that is, the compound of Formula I according to the invention. See, for example, Remington's Pharmaceutical Sciences, Mack Publishing Company, Easton, Pa., 17th edition, 1985.

The dosage of the compounds of Formula I to achieve a therapeutic effect will depend not only on such factors as the age, weight and sex of the patient and mode of administration, but also on the degree of potassium channel activating activity desired and the potency of the particular compound being utilized for the particular disorder of disease concerned. It is also contemplated that the treatment and dosage of the particular compound may be administered in unit dosage form and that the unit dosage form would be adjusted accordingly by one skilled in the art to reflect the relative level of activity. The decision as to the particular dosage to be employed (and the number of times to be administered per day) is within the discretion of the physician, and may be varied by titration of the dosage to the particular circumstances of this invention to produce the desired therapeutic effect.

A suitable dose of a compound of Formula I or pharmaceutical composition thereof for a mammal, including man, suffering from, or likely to suffer from any condition as described herein is an amount of active ingredient from about 0.01 μg/kg to 10 mg/kg body weight. For parenteral administration, the dose may be in the range of 0.01 μg/kg to 1 mg/kg body weight for intravenous administration. For oral administration, the dose may be in the range of 0.01 μg/kg to 5 mg/kg body weight. The active ingredient will preferably be administered in equal doses from one to four times a day. However, usually a small dosage is administered, and the dosage is gradually increased until the optimal dosage for the host under treatment is determined.

However, it will be understood that the amount of the compound actually administered will be determined by a physician, in the light of the relevant circumstances including the condition to be treated, the choice of compound of be administered, the chosen route of administration, the age, weight, and response of the individual patient, and the severity of the patient's symptoms.

The following examples are given by way of illustration and are not to be construed as limiting the invention in any way inasmuch as many variations of the invention are possible within the spirit of the invention.

### DESCRIPTION OF SPECIFIC EMBODIMENTS

Unless otherwise stated, solvents and reagents were used directly as obtained from commercial sources, and reactions were performed under a nitrogen atmosphere. Flash chromatography was conducted on Silica gel 60 (0.040-0.063 particle size; EM Science supply). ¹H NMR spectra were recorded on a Bruker DRX-500f at 500 MHz; a Bruker DPX-300B at 300 MHz; or a Varian Gemini 300 at 300 M Hz . The chemical shifts were reported in ppm on the δ scale relative to δTMS=0. The following internal references were used for the residual protons in the following solvents: CDCl₃ (δ_{H} 7.26), CD₃OD (δ_{H} 3.30) and DMSO-d₆ (δ_{H} 2.50). Standard acronyms were employed to describe the multiplicity patterns: s (singlet), d (doublet), t (triplet), q (quartet), m (multiplet), b (broad), app (apparent). The coupling constant (J) is in hertz. LC/MS was performed on a Shimadzu LC-10AS liquid chromatograph using a SPD-10 AV UV-VIS detector with Mass Spectrometry data determined using a Micromass LC Platform in positive electrospray ionization mode (ESI+). Mass Spectrometry (MS) data was obtained using a standard flow injection technique on a Micromass LC Platform in positive electrospray ionization mode (ESI+) unless otherwise noted. High resolution mass spectrometry (HRMS) data was obtained using a standard flow injection technique on a Finnigan MAT 900 mass spectrometer in electrospray ionization (ESI) mode. The analytical reverse phase HPLC method is as follows unless otherwise noted: Column YMC ODS-A C18 S7 (3.0×50 mm), Start % B=0, Final % B=100, Gradient Time=2 min, Flow rate 5 ml/min. Wavelength=220 nm, Solvent A=10% MeOH-90% H₂O-0.1% TFA, Solvent B=90% MeOH-10% H₂O-0.1% TFA; and Rₜ in min. Preparative reverse phase HPLC was performed on a Shimadzu LC-8A automated preparative HPLC system with detector (SPD-10AV UV-VIS) wavelength and solvent systems (A and B) the same as above except where otherwise noted. Melting points were determined using a standard Mel-temp apparatus at atmospheric pressure and are uncorrected.

### Preparation of Intermediates

Preparation 1

Preparation of the enaminone IIIa

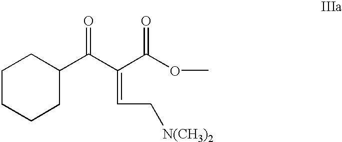

To a solution of methyl 3-cyclohexyl-3-oxopropionate, prepared according to the method of Taber et al, J. Amer. Chem. Soc., 1987, 109, 7488-7494, (3.46 g, 18.8 mmol) and DMF-Dimethyl sulfate adduct (5.0 g, 25.1 mmol, prepared by reacting a mixture of 1.05 mol of DMF and 1.0 mol of Me₂SO₄ at 40° C. for 4 hours and at room temperature for 48 hours) in dichloromethane (30 mL), was added triethylamine (3.8 mL, 27.3 mmol) at 0° C. The reaction mixture was stirred at room temperature for 16 hours and then washed consecutively with 10% tartaric acid and water and then the organic layer was dried over MgSO₄, filtered, and the filtrate concentrated in vacuo. The isolated crude material was purified by flash column chromatography (silica gel, 1:2 hexanes:ethyl acetate) to afford the intermediate enaminone of Formula IIIa, Preparation 1, as a light yellow solid (2.6 g).

Preparation 2

Preparation of 4-cyclohexyl-2-(methylthio)pyrimidine-5-carboxylic acid, methyl ester

To a mixture of 2-methylisothiourea sulfate (1.85 g, 6.66 mmol) and sodium acetate (2.28 g, 27.75 mmol) in DMF (20 ml) was added the enaminone IIIa (Preparation 1, 2.66 g, 11.1 mmol). The reaction mixture was heated at 80-90° C. for 16 hours. The reaction was cooled to room temperature and then diluted with water. The resulting precipitate was collected to afford the titled compound (1.9 g, 38%) as an off-white solid. MS m/e 267 (MH⁺). ¹H NMR (CDCl₃): δ 8.87(s, 1H), 3.91(s, 3H), 3.59 (m, 1H), 2.60(s, 3H), 1.30-1.86(m, 10H).

Preparation 3

Preparation of 4-cyclohexyl-2-(methylsulfinyl)pyrimidine-5-carboxylic acid, methyl ester

To a solution of 4-cyclohexyl-2-(methylthio)pyrimidine-5-carboxylic acid, methyl ester (Preparation 2,1.5 g, 5.63 mmol) in dichloromethane (30 mL), 3-chloroperoxybenzoic acid (1.17 g, 6.76 mmol) was added at −78° C. The reaction mixture was stirred in an ice bath for 3 hours. The reaction mixture was washed consecutively with saturated sodium bicarbonate solution and brine. The organic layer was then dried over MgSO₄, filtered, and the filtrate was concentrated in vacuo to give the titled compound (1.4 g, 88%) as a yellow solid. MS m/e 283 (MH⁺). ¹H NMR (CDCl₃): δ 9.19(s, 1H), 3.99(s, 3H), 3.61(m, 1H), 2.96(s, 3H), 1.27-1.84(m, 10H).

Preparation 4

Preparation of 4-cyclohexyl-2-(morpholin-1-yl)pyrimid ine-5-carboxylic acid, methyl ester

To a solution of 4-cyclohexyl-2-(methylsulfinyl)pyrimidine-5-carboxylic acid, methyl ester (Preparation 3, 0.4 g, 1.41 mmol) in THF (3 mL), was added morpholine (0.247 mL, 2.83 mmol). The reaction mixture was heated under reflux for 3 hours. The reaction mixture was then concentrated in vacuo to remove solvent and excess amine. The resultant residue was washed with saturated sodium bicarbonate solution to give the titled compound (0.4 g, 93%) as a white solid. MS m/e 306 (MH⁺).

Preparation 5

Preparation of 4-cyclohexyl-2-(morpholin-1-yl)pyrimidine-5-carboxylic acid

A solution of 4-cyclohexyl-2-(morpholin-1-yl)pyrimidine-5-carboxylic acid, methyl ester (Preparation 4, 0.21 g, 0.68 mmol) in 10 N sodium hydroxide (5 mL) and methanol (5 mL) was heated under reflux for 3 hours. The solvents were removed in vacuo. The resultant aqueous residue was neutralized with 1 N HCl to pH 7. The pure titled compound (0.15 g, 76%) was collected as a white solid: MS m/e 292 (MH⁺).

Preparation 6

Preparation of 2,4-Dichloro-N-[[4-(trifluoromethyl)phenyl]methyl]pyrimidine-5-carboxamide

A suspension of 2,4-dihydroxypyrimidine-5-carboxylic acid (20.0 g, 0.128 mol) in POCl₃ (700 mL) was refluxed for 11 hours, cooled to 23° C. and treated with PCl₅. The reaction mixture was then refluxed for 16 hours. The mixture was cooled to 23° C. and concentrated under reduced pressure to give a thick syrup. Traces of volatile phosphorus derivatives were co-distilled twice with toluene (2×250 mL) leaving 18.9 g (70%) of a thick red syrup [as described by E. L. Stogryn in J. Med. Chem., 1972, 15(2), 200-201]. The crude material was used without further purification. Crude 2,4-dichloro-5-pyrimidinecarbonyl chloride (10 g) was diluted in anhydrous tetrahydrofuran (200 mL), cooled to −78° C. then triethylamine (20 mL, 0.142 mol) was added in one portion. The cold mixture was then treated dropwise with 4-(trifluoromethyl)benzylamine (6.74 mL, 0.047 mol), stirred for 1 hour and then diluted with HCl (0.5 N, 200 mL). The reaction mixture was then extracted with ethyl acetate, the combined organic layer was dried over MgSO₄, filtered, and the filtrate concentrated in vacuo to afford 11.9 g (71%) of crude titled compound. Recrystallization of the crude product from tetrahydrofuran-hexanes afforded the pure titled compound as a crystalline solid.

Preparation 7

Preparation of 2-Chloro-4-(pyrrolidin-1-yl)-N-[[4-(trifluoromethyl)phenyl]methyl]pyrimidine-5-carboxamide

A cold (0° C.) solution of 2,4-dichloro-N-[[4-(trifluoromethyl)phenyl]methyl]pyrimidine-5-carboxamide (Preparation 6, 8.35 g, 0.238 mmol) and triethylamine (5 mL, 35.7 mmol) in anhydrous tetrahydrofuran (60 mL) was treated dropwise with pyrrolidine (2.2 mL, 26. 2 mmol). The mixture was stirred at 0° C. for 1.5 hours and then quenched by addition of HCl (1 N, 40 mL) and H₂0 (100 mL). The resulting mixture was extracted several times with ethyl acetate. The organic extracts were dried over Na₂SO₄, filtered, and the filtrate was concentrated in vacuo leaving 7.9 g (86%) of crude title compound. Recrystallization of the crude product from tetrahydrofuran-hexanes afforded pure title compound as a solid: mp 170-171° C.; ¹H NMR (CDCl₃) δ 8.08 (s, pyrimidine H, 1H), 7.65 (d, J=8 Hz, 2H), 7.51 (d, J=8 Hz, 2H), 6.52 (bs, NH, 1H), 4.66 (d, J =5.9 Hz, benzylic H's 2H), 3.46 (bt, J=6.7 Hz, NCH²CH², 4H), 1.93 (bt, J =6.7 Hz, NCH₂CH₂, 4H); IR 3273, 1654, 1585, 1539, 1389, 1326, 1212, 1175, 1111, 1066, 1011 cm^{31 1}; Anal. Calcd. for C₁₇H₁₆ClF₃N₄O: C, 53.07; H, 4.19; N, 14.56. Found: C, 53.34; H, 4.28; N, 14.36; HRMS/ESI C₁₇H₁₇OF₃N₄³⁵Cl (M+H)⁺; 385.10431 found: 385.10450.

### PREPARATION OF COMPOUNDS OF FORMULA I

The following examples illustrate the preparation of the compounds of Formula I by following the general procedures described herein.

### EXAMPLE 1

### 4-Cyclohexyl-2-(morpholin-1-yl)-N-[(4-fluorophenyl)methyl]pyrimidine-5-carboxamide

To a solution of 4-cyclohexyl-2-(morpholin-1-yl)pyrimidine-5-carboxylic acid (Preparation 5,23.3 mg, 0.08 mmol) in dichloromethane (2 mL) was added polymer supported 1-(3-dimethylaminopropyl)-3-ethylcarbodiimide resin (P-EDC resin, 457 mg, 0.64 mmol) and 4-fluorobenzylamine (18.3 μl, 0.16 mmol). The reaction mixture was stirred at room temperature for 16 hours. The resin was filtered off and the solvent was removed in vacuo. The resultant residue was purified by preparative HPLC and the titled compound isolated as the TFA salt: MS m/e 399 (MH^{+).}

### EXAMPLES 2-18

Examples 2 through 18 were prepared according to the general method described and depicted below. The intermediates of Formula VII were obtained from the corresponding appropriate starting materials as described in Scheme 1 and by methods as further described for Preparations 1-5.

### General Procedure for the Preparation of 2-(Substituted Amino)Pyrimidine-5-Carboxamide Compounds of Formula Ia (Examples 2-18)

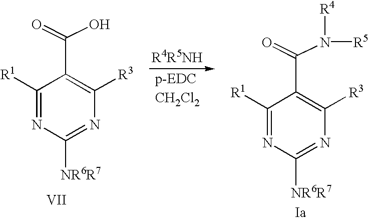

To a solution of intermediate of Formula VII (0.08 mmol) in dichloromethane (2 mL), polymer supported 1-(3-dimethylaminopropyl)-3-ethylcarbodiimide resin (457 mg, 0.64 mmol) and an appropriate primary or secondary amine of formula HNR⁴R⁵ (0.16 mmol) were added. The reaction mixture was stirred at room temperature for 16 hours. The resin was filtered off and the solvent was removed in vacuo. The resultant residue was purified by preparative HPLC to afford the compound of Formula Ia which is isolated as the TFA salt. The compounds of Examples 2 through 18 were prepared from the corresponding intermediate of Formula VII by following this general procedure.

| Ex- | | |
|---|---|---|
| am- | | |
| ple | | Mass |
| No. | Chemical Name | Spectrum m/e |
| 2 | 4-Cyclohexyl-2-(morpholin-1-yl)-N- | 381 (MH⁺) |
| | (phenylmethyl)pyrimidine-5-carboxamide | |
| 3 | 4-Cyclohexyl-2-(piperidin-1-yl)-N-[(4- | 397 (MH⁺) |
| | fluorophenyl)methyl]pyrimidine-5-carboxamide | |
| 4 | 4-Cyclohexyl-2-(pyrrolidin-1-yl)-N-[(4- | 383 (MH⁺) |
| | fluorophenyl)methyl]pyrimidine-5-carboxamide | |
| 5 | 4-Cyclohexyl-2-[ethyl(phenylmethyl)amino]-N- | 435 (MH⁺) |
| | (cyclohexylmethyl)pyrimidine-5-carboxamide | |
| 6 | 4-Cyclohexyl-2-[ethyl(phenylmethyl)amino]-N- | 429 (MH⁺) |
| | (phenylmethyl)pyrimidine-5-carboxamide | |
| 7 | (4-Cyclohexyl-2-[ethyl(phenylmethyl)amino]-N- | 447 (MH⁺) |
| | [(4-fluorophenyl)methyl]pyrimidine-5- | |
| | carboxamide | |
| 8 | (±)-4-Cyclohexyl-2-[(2-methylpiperidin)-1-yl]-N- | 399 (MH⁺) |
| | (cyclohexylmethyl)pyrimidine-5-carboxamide | |
| 9 | 4-(1-Propyl)-2-(pyrrolidin-1-yl)-N-[(4- | 343 (MH⁺) |
| | fluorophenyl)methyl]pyrimidine-5-carboxamide | |
| 10 | 4-(1-Propyl)-2-(piperidin-1-yl)-N-[(4- | 357 (MH⁺) |
| | fluorophenyl)methyl]pyrimidine-5-carboxamide | |
| 11 | 4-(2-Propyl)-2-(pyrrolidin-1-yl)-N-[(4- | 343 (MH⁺) |
| | fluorophenyl)methyl]pyrimidine-5-carboxamide | |
| 12 | 4-(2-Propyl)-2-(piperidin-1-yl)-N-[(4- | 357 (MH⁺) |
| | fluorophenyl)methyl]pyrimidine-5-carboxamide | |
| 13 | 4-(2-Propyl)-2-(morpholin-1-yl)-N-[(4- | 359 (MH⁺) |
| | fluorophenyl)methyl]pyrimidine-5-carboxamide | |
| 14 | 4-(2-Propyl)-2-[ethyl(phenylmethyl)amino]-N- | 389 (MH⁺) |
| | (phenylmethyl)pyrimidine-5-carboxamide | |
| 15 | 4-(2-Fluorophenyl)-2-(pyrrolidin-1-yl)-N-[[4- | 445 (MH⁺) |
| | (trifluoromethyl)phenyl]methyl]pyrimidine-5- | |
| | carboxamide | |
| 16 | 4-(2-Fluorophenyl)-2-(morpholin-1-yl)-N-[[4- | 461 (MH⁺) |
| | (trifluoromethyl)phenyl]methyl]pyrimidine-5- | |
| | carboxamide | |
| 17 | 4-(4-Fluorophenyl)-2-(pyrrolidin-1-yl)-N-[[3- | 445 (MH⁺) |
| | (trifluoromethyl)phenyl]methyl]pyrimidine-5- | |
| | carboxamide | |
| 18 | 4-(2-Fluorophenyl)-2-(morpholin-1-yl)-N-[[3- | MS m/e |
| | (trifluoromethyl)phenyl]methyl]pyrimidine-5- | 461 |
| | carboxamide | |

### EXAMPLE 19

### 2-(Pyrrolidin-1-yl)-4-(trifluoromethyl)-N-[[4-trifluoromethyl)phenyl]methyl]pyrimidine-5-carboxamide

To a solution of 2-chloro-4-(trifluoromethyl)pyrimidine-5-carbonyl chloride (0.74 g, 3.0 mmol) in dichloromethane (5 mL), was added saturated sodium bicarbonate (5 mL) and 4-(trifluoromethyl)benzylamine (0.58 g, 3.3 mmol). The reaction mixture was stirred at room temperature for 3 hours. The precipitated white solid of 2-chloro-4-(trifluoromethyl)-N-[[4-(trifluoromethyl)phenyl]methyl]pyrimidine-5-carboxamide was collected by filtration and then dissolved in acetonitrile (10-15 mL). Potassium carbonate (0.62 g, 4.5 mmol) and pyrrolidine (0.43 g, 6 mmol)) were added. The reaction mixture was stirred at room temperature overnight. The inorganic salts were filtered off and the filtrate was concentrated in vacuo to provide the pure titled compound MS m/e 419 (MH⁺). ¹H NMR (DMSO-d₆): δ 9.10 (t, J=5.9 Hz, 1H), 8.68 (s, 1H), 7.71 (d, J=8.1 Hz, 2H), 7.55 (d, J=8.0 Hz, 2H), 4.51 (d, J=6.1 Hz, 2H), 3.5-3.55 (m, br, 4H), 1.93-1.98 (m, 4H)

### EXAMPLES 20-30

### General Procedure for the Preparation of 2-(substituted Amino)pyrimidine-5-carboxamide compounds of Formula Ia' (Examples 20-30)

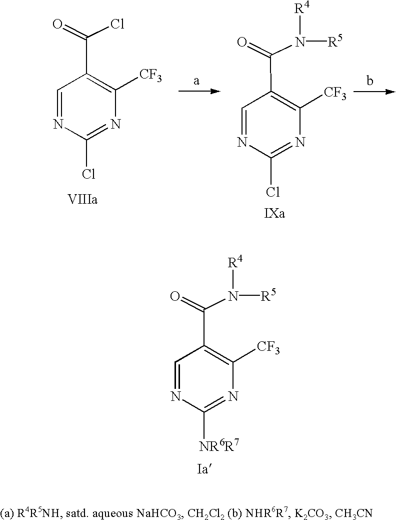

To a solution of 2-chloro-4-(trifluoromethyl)pyrimidine-5-carbonyl chloride (VIIIa , 0.74 g, 3.0 mmol) in dichloromethane (5 mL), was added saturated sodium bicarbonate (5 mL) and an appropriate amine of formula R⁴R⁵NH (3.3 mmol). The reaction mixture was stirred at room temperature for 3 hours. The precipitated solid of the 2-chloro-4-trifluoromethyl)-N-pyrimidine-5-carboxamide derivative of Formula IXa was collected by filtration and then dissolved in acetonitrile (10-15 mL). Potassium carbonate (0.62 g, 4.5 mmol) and an appropriate amine of formula NHR⁶R⁷ (6 mmol)) was added. The reaction mixture was stirred at room temperature overnight. The inorganic salts were filtered off and the filtrate was concentrated in vacuo to provide the pure titled compound.

The compounds of Examples 20 through 30 were prepared from the compound of Formula VIIIa using the appropriate amines following the general procedure described above.

| Ex- | | |
|---|---|---|
| am- | | Mass |
| ple | | Spectrum |
| No. | Chemical Name | m/e |
| 20 | 4-(Trifluoromethyl)-2-(pyrrolidin-1-yl)-N-[[3- | 419 (MH⁺) |
| | (trifluoromethyl)phenyl]methyl]pyrimidine-5- | |
| | carboxamide | |
| 21 | 4-(Trifluoromethyl)-2-(pyrrolidin-1-yl)-N-[(4- | 385 (MH⁺) |
| | chlorophenyl)methyl]pyrimidine-5-carboxamide | |
| 22 | 4-(Trifluoromethyl)-2-(morpholin-4-yl)-N-[(4- | 401 (MH⁺) |
| | chlorophenyl)methyl]pyrimidine-5-carboxamide | |
| 23 | 4-(Trifluoromethyl)-2-(hexamethyleneimin-1-yl)- | 393 (MH⁺) |
| | N-[(4-methylphenyl)methyl]pyrimidine-5- | |
| | carboxamide | |
| 24 | 4-(Trifluoromethyl)-2-(pyrrolidin-1-yl)-N-[[4- | 435 (MH⁺) |
| | (trifluoromethoxy)phenyl]methyl]pyrimidine-5- | |
| | carboxamide | |
| 25 | 4-(Trifluoromethyl)-2-(morpholin-1-yl)-N-[[4- | 451 (MH⁺) |
| | (trifluoromethoxy)phenyl]methyl]pyrimidine-5- | |
| | carboxamide | |
| 26 | 4-(Trifluoromethyl)-2-(piperidin-1-yl)-N-[[4- | 449 (MH⁺) |
| | (trifluoromethoxy)phenyl]methyl]pyrimidine-5- | |
| | carboxamide | |
| 27 | 4-(Trifluoromethyl)-2-(diethylamino)-N-[[4- | 437 (MH⁺) |
| | (trifluoromethoxy)phenyl]methyl]pyrimidine-5- | |
| | carboxamide | |
| 28 | 4-(Trifluoromethyl)-2-(pyrrolidin-1-yl)-N-[(4- | 431 (MH⁺) |
| | bromophenyl)methyl]pyrimidine-5-carboxamide | |
| 29 | 4-(Trifluoromethyl)-2-(pyrrolidin-1-yl)-N-[[4- | 394 (MH⁺) |
| | (dimethylamino)phenyl]methyl]pyrimidine-5- | |
| | carboxamide | |
| 30 | 4-(Trifluoromethyl)-2-(morpholin-1-yl)-N-[[4- | 467 (MH⁺) |
| | (trifluoromethylthio)phenyl]methyl]pyrimidine-5- | |
| | carboxamide | |

### EXAMPLE 31

### 2-Amino-4-(pyrrolidin-1-yl)-N-[[4-(trifluoromethyl)phenyl]methyl]pyrimidine-5-carboxamide

A solution of 2-chloro-4-(pyrrolidin-1-yl)-N-[[4-(trifluoromethyl)phenyl]methyl]pyrimidine-5-carboxamide (Preparation 7, 0.60 g, 1.56 mmol) in 1-methyl-2-pyrrolidinone (25 mL) was cooled to approximately 0° C. and saturated with ammonia gas in a steel bomb. The steel bomb was sealed and heated at 120° C. for 24 hours. After cooling to 23° C. the mixture was diluted with water and extracted with ethyl acetate. The combined organic extract was washed with water, dried over MgSO₄, filtered and the filtrate was concentrated under reduced pressure. The residue was triturated with diethyl ether and collected by filtration to afford the titled compound as a tan solid (0.41 g, 72%): mp 227-228° C.; ¹H NMR (DMSO- d₆) δ 8.76 (t, J=6.13 Hz, NH, 1H), 7.90 (s, pyrimidine H, 1H), 7.71 (d, J=8.1 Hz, 2H), 7.54 (d, J=8.1 Hz, 2H), 6.26 (s, NH₂, 2H), 4.44 (d, J=6.1 Hz, benzylic H's 2H), 3.27 (bt, J=6.5 Hz, NCH₂CH₂, 4H), 1.77 (bt, J=6.5 Hz, NCH₂CH₂, 4H); IR 3476, 3279, 1629, 1585, 1529, 1456, 1377, 1330, 1160, 1110, 1069 cm^{31 1}.

### EXAMPLES 32-46

### General procedure for the preparation of 2-[Alkyl(aryl)amino]-4-(pyrrolidin-1-yl)-N-[[4-(trifluoromethyl)phenyl]methyl]pyrimidine-5-carboxamides (Examples 32-46)

To a solution of 2-chloro-4-(pyrrolidin-1-yl)-N-[[4-(trifluoromethyl)phenyl]methyl]pyrimidine-5-carboxamide (Preparation 7, 9.6 mg, 0.025 mmol) in 1-methyl-2-pyrrolidone (0.5 n mL) was added 1.0 M solution of an appropriate amine derivative of the formula HNR⁶R⁷ (0.125 mL, 0.125 mmol, 5 eq.) in 1-methyl-2-pyrrolidinone. The resulting mixture was heated for 18 hours at a temperature of 100° C. to 135° C. (100° C. when HNR⁶R⁷ is an alkylamine; 135° C. when HNR⁶R⁷ is an aniline derivative). The crude mixture was purified by HPLC (PRIMESPHERE C18,21.1 mm×100 mm column); mobile phase: A 10/90 CH₃CN/H₂0+5 mMol NH₄OAc, B 90/10 CH₃CN/H₂0+5 mMol NH₄OAc; gradient: 40% to 0% of A over 5 minutes; detector: UV, 220 nM; Flow rate: 20.0 mL/min. Purity of samples was analyzed by LCMS: HPLC (the first nine compounds) (LUNA C8, 5μ, 4.6 mm×30 mm column); mobile phase: A 10/90 CH₃CN/H₂0+5 mMol NH₄OAc, B 90/10 CH₃CN/H₂0+5 mMol NH₄OAc; gradient: 100% to 0% of A over 4 minutes; detector: UV, 250 nM; Flow rate: 4.0 mL/min; (the last seven compounds) (Primesphere C18-HC, 4.6 mm×30 mm column); mobile phase: A 10/90 CH₃CN/H₂0+5 mMol NH₄OAc, B 90/10 CH₃CN/H₂0+5 mMol NH₄OAc; gradient: 100% to 0% of A over 3 minutes; detector: UV, 250 nM; Flow rate: 4.0 mL/min.

Examples 32 through 46 were prepared by following the general procedure described above.

| Ex- | | |
|---|---|---|
| am- | | Mass |
| ple | | Spectrum |
| No. | Chemical Name | m/e |
| 32 | 2-(3-Pentylamino)-4-(pyrrolidin-1-yl)-N-[[4- | 436 (MH⁺) |
| | (trifluoromethyl)phenyl]methyl]pyrimidine-5- | |
| | carboxamide | |
| 33 | 2-[Ethyl(2-hydroxyethyl)amino]-4-(pyrrolidin-1- | 438 (MH⁺) |
| | yl)-N-[[4-(trifluoromethyl)phenyl]methyl]- | |
| | pyrimidine-5-carboxamide | |
| 34 | 2-[Methyl(1-pentyl)amino]-4-(pyrrolidin-1-yl)-N- | 450 (MH⁺) |
| | [[4-(trifluoromethyl)phenyl]methyl]pyrimidine-5- | |
| | carboxamide | |
| 35 | 2-4-Bis-(pyrrolidin-1-yl)-N-[[4-(trifluoromethyl) | 420 (MH⁺) |
| | phenyl]methyl]pyrimidine-5-carboxamide | |
| 36 | 2-(Cyclopentylamino)-4-(pyrrolidin-1-yl)-N-[[4- | 434 (MH⁺) |
| | (trifluoromethyl)phenyl]methyl]pyrimidine-5- | |
| | carboxamide | |
| 37 | 2-(Phenylmethylamino)-4-(pyrrolidin-1-yl)-N-[[4- | 456 (MH⁺) |
| | (trifluoromethyl)phenyl]methyl]pyrimidine-5- | |
| | carboxamide | |
| 38 | 2-[Methyl(phenylmethyl)amino]-4-(pyrrolidin-1- | 470 (MH⁺) |
| | yl)-N-[[4-(trifluoromethyl)phenyl]methyl]- | |
| | pyrimidine-5-carboxamide | |
| 39 | 2-[(4-Chlorophenyl)methylamino]-4-(pyrrolidin- | 490 (MH⁺) |
| | 1-yl)-N-[[4-(trifluoromethyl)phenyl]methyl]- | |
| | pyrimidine-5-carboxamide | |
| 40 | 2-[Methyl(2-propyn-1-yl)amino]-4-(pyrrolidin-1- | 418 (MH⁺) |
| | yl)-N-[[4-(trifluoromethyl)phenyl]methyl]- | |
| | pyrimidine-5-carboxamide | |
| 41 | 2-(Diethylamino)-4-(pyrrolidin-1-yl)-N-[[4- | 422 (MH⁺) |
| | (trifluoromethyl)phenyl]methyl]pyrimidine-5- | |
| | carboxamide | |
| 42 | 2-[Methyl[(4-trifluoromethoxy)phenyl]amino]-4- | 540 (MH⁺) |
| | (pyrrolidin-1-yl)-N-[[4-(trifluoromethyl) | |
| | phenyl]methyl]pyrimidine-5-carboxamide | |
| 43 | 2-[Methyl(4-nitrophenyl)amino]-4-(pyrrolidin-1- | 501 (MH⁺) |
| | yl)-N-[[4-(trifluoromethyl)phenyl]methyl]- | |
| | pyrimidine-5-carboxamide | |
| 44 | 2-[(2-Propyn-1-yl)amino]-4-(pyrrolidin-1-yl)-N- | 404 (MH⁺) |
| | [[4-(trifluoromethyl)phenyl]methyl]pyrimidine-5- | |
| | carboxamide | |
| 45 | 2-[(3-Chlorophenyl)methylamino]-4-(pyrrolidin- | 490 (MH⁺) |
| | 1-yl)-N-[[4-(trifluoromethyl)phenyl]methyl]- | |
| | pyrimidine-5-carboxamide: | |
| 46 | 2-[(3,4-Dichlorophenyl)methylamino]-4- | 524 (MH⁺) |
| | (pyrrolidin-1-yl)-N-[[4-(trifluoromethyl) | |
| | phenyl]methyl]pyrimidine-5-carboxamide | |

### EXAMPLE 47

### 2-(4-Fluorophenoxy)-4-(pyrrolidin-1-yl)-N-[[4-trifluoromethyl)phenyl]methyl]pyrimidine-5-carboxamide

To a solution of 2-chloro-4-(pyrrolidin-1-yl)-N-[[4-trifluoromethyl)phenyl]methyl]pyrimidine-5-carboxamide (Preparation 7, 9.6 mg, 0.025 mmol) in 1-methyl-2-pyrrolidinone (0.5 mL) was added a 1.0 M solution of 4-fluorophenol (0.125 mL, 0.125 mmol, 5 eq.) in 1-methyl-2-pyrrolidinone and a solution of potassium tert-butoxide in tetrahydrofuran (1.0 M, 0.125 mL, 0.125 mmol, 5 eq.). The resulting mixture was heated at 85° C. for 15 hours. After cooling to approximately 23° C., the mixture was quenched by addition of an aqueous solution of NaH₂PO₄ (1.0 M, 0.25 mL) and filtered on PTFE filter prior purification by HPLC (PRIMESPHERE C18-HC, 21.2 mm×100 mm column); mobile phase: A 10/90 CH₃CN/H₂0+5 mMol NH₄OAc, B 90/10 CH₃CN/H₂0+5 mMol NH₄OAc; gradient: 40% to 0% of A over 5 minutes; detector: UV, 220 nM; Flow rate: 20.0 mL/min. Purity of the sample was analyzed by LCMS: HPLC (Primesphere C18-HC, 4.6 mm×30 mm column); mobile phase: A 10/90 CH₃CN/H₂0+5 mMol NH₄OAc, B 90/10 CH₃CN/H₂0+5 mMol NH₄OAc; gradient: 100% to 0% of A over 3 minutes; detector: UV, 250 nM; Flow rate: 4.0 mL/min. MS m/e 461 (MH⁺); ¹H NMR (CDCl₃) δ 8.22 (s, pyrimidine H, 1H), 7.61 (d, J=7.8 Hz, 2H), 7.51 (d, J=7.8 Hz, 2H), 7.38 (bs, NH, 1H), 7.15-6.95 (m, 4H), 4.64 (d, J=3.1 Hz, benzylic H's 2H), 3.35 (bt, J=6.3 Hz, NCH₂CH₂, 4H), 1.86 (bs, NCH₂CH₂, 4H).

### EXAMPLE 48

### 2-[(2-Methoxy)phenoxy]-4-(Pyrrolidin-1-yl)-N-[[4-(trifluoromethyl)phenyl]methyl]pyrimidine-5-carboxamide

To a solution of 2-chloro-4-(pyrrolidin-1-yl)-N-[[4-(trifluoromethyl)phenyl]methyl]pyrimid ine-5-carboxamide (Preparation 7, 9.6 mg, 0.025 mmol) in 1-methyl-2-pyrrolidinone (0.5 mL) was added a 1.0 M solution of 2-methoxyphenol (0.125 mL, 0.125 mmol, 5 eq.) in 1-methyl-2-pyrrolidinone and a solution of potassium tert-butoxide in tetrahydrofuran (1.0 M, 0.125 ml, 0.125 mmol, 5 eq.). The resulting mixture was heated at 85° C. for 15 hours. After cooling to approximately 23° C., the mixture was quenched by addition of an aqueous solution of NaH₂PO₄ (1.0 M, 0.25 mL) and filtered on PTFE filter prior purification by HPLC (PRIMESPHERE C18-HC, 21.2 mm×100 mm column); mobile phase: A 10/90 CH₃CN/H₂0+5 mMol NH₄OAc, B 90/10 CH₃CN/H₂0+5 mMol NH₄OAc; gradient: 40% to 0% of A over 5 minutes; detector: UV, 220 nM; Flow rate: 20.0 mL/min. Purity of the sample was analyzed by LCMS: HPLC (Primesphere C18-HC, 4.6 mm×30 mm column); mobile phase: A 10/90 CH₃CN/H₂0+5 mMol NH₄OAc, B 90/10 CH₃CN/H₂0+5 mMol NH₄OAc; gradient: 100% to 0% of A over 3 minutes; detector: UV, 250 nM; Flow rate: 4.0 mL/min. MS m/e 473 (MH⁺).

### EXAMPLES 49-51

The 2-alkoxy-4-(pyrrolidin-1-yl)-N-[[4-(trifluoromethyl) phenyl]methyl]pyrimidine-5-carboxamide derivatives (Examples 49-51) were prepared by reacting 2-chloro-4-(pyrrolidin-1-yl)-N-[[4-(trifluoromethyl)phenyl]methyl]pyrimidine-5-carboxamide with the sodium salt of an appropriate alcohol derivative as described in the following general procedure.

General Procedure for Examples 49-51

To a solution of 2-chloro-4-(pyrrolidin-1-yl)-N-[[4-(trifluoromethyl)phenyl]methyl]pyrimidine-5-carboxamide (Preparation 7, 19.2 mg, 0.050 mmol) in dioxane (0.6 mL) was added a 1.0 M solution of an appropriate alcohol (0.40 mL, 0.40 mmol, 8 eq.) in dioxane followed by a 1.0 M solution of sodium hexamethyldisilazide (NaHMDS) in tetrahydrofuran (0.250 mL, 0.250 mmol, 5 eq.). The resulting mixture was heated at 70° C. for 2 hours. After cooling to 23° C., the mixture was quenched by addition of an aqueous solution of NH₄Cl (1.0 N, 0.40 mL) and filtered through a PTFE filter. The reaction vessel was rinsed with methanol and the resulting solution was filtered as well. The crude filtrates were combined and purified by HPLC (PRIMESPHERE C18-HC, 21.2 mm×100 mm column); mobile phase: A 10/90 CH₃CN/H₂0+5 mMol NH₄OAc, B 90/10 CH₃CN/H₂0+5 mMol NH₄OAc; gradient: 40% to 0% of A over 5 minutes; detector: UV, 220 nM; Flow rate: 20.0 mL/min. Purity of each sample was analyzed by LCMS: HPLC (YMC ODS-A C18, 4.6 mm×33 mm column); mobile phase: A 10/90 CH₃CN/H₂0+5 mMol NH₄OAc, B 90/10 CH₃CN/H₂0+5 mMol NH₄OAc; gradient: 100% to 0% of A over 3 minutes; detector: UV, 220 nM; Flow rate: 4.0 mL/min.

### EXAMPLE 49

### 2-(Propyn-3-yloxy)-4-(pyrrolidin-1-yl)-N-[[4-(trifluoromethyl)phenyl]methyl]pyrimidine-5-carboxamide

¹H NMR (CDCl₃) δ 8.19 (s, pyrimidine H, 1H), 8.0 (bs, NH, 1H), 7.58 (d, J =8.1 Hz, 2H), 7.51 (d, J=8.1 Hz, 2H), 4.92 (d, J=2.5 Hz,OCH₂, 2H), 4.60 (d, J=5.8 Hz, benzylic 's H, 2H), 3.47 (bt, J=6.6 Hz, NCH₂CH₂, 4H), 2.44 (d, J=2.5 Hz, ynyl H, 1H), 1.87 (bt, J=6.6 Hz, NCH₂CH₂, 4H); HRMS/ESI C₂₀H₂₀O₂F₃N₄ (M+H)⁺; 405.15384 found: 405.15480.

### EXAMPLE 50

### 2-[(2-Thienyl)methoxy]-4-(pyrrolidin-1-yl)-N-[[4-(trifluoromethyl)phenyl]methyl]pyrimidine-5-carboxamide

¹H NMR (CDCl₃) δ 8.10 (s, pyrimidine H, 1H), 7.56 (d, J=8.1 Hz, 2H), 7.46 (d, J=8.1 Hz, 2H), 7.27 (d, J=1.5 Hz, 1H), 7.08 (d, J=3.5 Hz, 1H), 6.95 (dd, J=1.5 Hz, J=3.5 Hz, 1H), 5.49 (s, OCH₂, 2H), 4.57 (d, J=5.8 Hz, benzylic 's H, 2H), 3.47 (bs, NCH₂CH₂, 4H), 1.87 (bs, NCH₂CH₂, 4H); HRMS/ESI C₂₂H₂₂O₂F₃N₄S (M+H)⁺; 463.14157 found: 463.14350.

### EXAMPLE 51

### 2-[1-(4-Fluorophenyl)ethoxy]-4-(pyrrolidin-1-yl)-N-[[4-(trifluoromethyl)phenyl]methyl]pyrimidine-5-carboxamide

¹H NMR (CDCl₃) δ 8.20 (s, pyrimidine H, 1H), 7.58 (d, J=8.3 Hz, 2H), 7.49 (d, J=8.3 Hz, 2H), 7.34 (dd, J=5.4 Hz, J=2.0 Hz, 2H), 6.99 (t, J=8.7 Hz, 2H), 6.02 (q, J=6.6 Hz, PhCH(CH₃)O, 1H), 4.49 (d, J=6.1 Hz, PhCH₂N, 2H), 3.5-3.25 (m, NCH₂CH₂, 4H), 1.95-1.80 (m, NCH₂CH₂, 4H) 1.63 (s, J=6.6 Hz, PhCH(CH₃)O, 3H); HRMS/ESI C₂₅H₂₅O₂F₄N₄ (M+H)⁺; 489.19138 found: 489.19070.

### EXAMPLES 52-55

The 2-aryl-4-(pyrrolidin-1-yl)-N-[[4-(trifluoromethyl)phenyl]-methyl]pyrimidine-5-carboxamide derivatives of Examples 52-55 were prepared by Pd(0) mediated coupling of 2-chloro-4-(pyrrolidin-1-yl)-N-[[4-(trifluoromethyl)phenyl]methyl]pyrimidine-5-carboxamide with appropriate aryl boronic acid derivative as described in the following general procedure.

General Procedure for Examples 52-55

To a solution of 2-chloro-4-(pyrrolidin-1-yl)-N-[[4-(trifluoromethyl)phenyl]methyl]pyrimidine-5-carboxamide (Preparation 7, 20.0 mg, 0.052 mmol) in 1-methyl-2-pyrrolidinone (1.0 mL) was added an appropriate boronic acid derivative [3,4-dimethoxyphenylboronic acid, 2-methoxyphenylboronic acid, 3-thienylboronic acid and 2-thienylboronic acid for Examples 52-55, respectively (1.17 mmol, 2.25 eq.)] followed by an aqueous solution of NaHCO₃ (2.0 M, 0.10 mL). The resulting mixture was flushed with argon prior to the addition of tetrakis triphenylphosphine palladium (Pd(PPh₃)₄) (0.003 g) and then heated at 110° C. for 2 hours. After cooling at 23° C., the mixture was purified by HPLC (PRIMESPHERE C18-HC, 21.2 mm×100 mm column); mobile phase: A 10/90 CH₃CN/H₂0+5 mMol NH₄OAc, B 90/10 CH₃CN/H₂0+5 mMol NH₄OAc; gradient: 70% to 0% of A over 8 minutes; detector: UV, 220 nM; Flow rate: 20.0 mL/min. Purity of each sample was analyzed by LCMS: HPLC (Primesphere C 18-HC, 6 mm×30 mm column); mobile phase: A 10/90 CH₃CN/H₂0+5 mMol NH₄OAc, B 90/10 CH₃CN/H₂0+5 mMol NH₄OAc; gradient: 100% to 0% of A over 3 minutes; detector: UV, 250 nM; Flow rate: 4.0 mL/min.

| Ex- | | |
|---|---|---|
| am- | | Mass |
| ple | | Spectrum |
| No. | Chemical Name | m/e |
| 52 | 2-(3,4-Dimethoxyphenyl)-4-(pyrrolidin-1-yl)-N- | 487 (MH⁺) |
| | [[4-(trifluoromethyl)phenyl]methyl]pyrimidine-5- | |
| | carboxamide | |
| 53 | 2-(2-Methoxyphenyl)-4-(pyrrolidin-1-yl)-N-[[4- | 457 (MH⁺) |
| | (trifluoromethyl)phenyl]methyl]pyrimidine-5- | |
| | carboxamide | |
| 54 | 2-(3-Thienyl)-4-(pyrrolidin-1-yl)-N-[[4- | 433 (MH⁺) |
| | (trifluoromethyl)phenyl]methyl]pyrimidine-5- | |
| | carboxamide | |
| 55 | 2-(2-Thienyl)-4-(pyrrolidin-1-yl)-N-[[4- | 433 (MH⁺) |
| | (trifluoromethyl)phenyl]methyl]pyrimidine-5- | |
| | carboxamide | |

### EXAMPLES 56-64

The 2-alkyl(aryl)thio-4-(pyrrolidin-1-yl)-N-[[4-trifluoromethyl)phenyl]methyl]pyrimidine-5-carboxamide derivatives (Examples 56-64) were prepared by reacting 2-chloro-4-(pyrrolidin-1 -yl)-N-[[4-trifluoromethyl)phenyl]methyl]pyrimidine-5-carboxamide with the potassium salt of an appropriate thiol derivative as described in the following general procedure.

General Procedure for Examples 56-64

To a solution of 2-chloro-4-(pyrrolidin-1-yl)-N-[[4-(trifluoromethyl) phenyl]methyl]pyrimidine-5-carboxamide (Preparation 7, 9.6 mg, 0.025 mmol) in 1-methyl-2-pyrrolidinone (0.5 mL) was added 1.0 M solution of an appropriate thiol derivative (0.125 mL, 0.125 mmol, 5 eq.) in 1-methyl-2-pyrrolidinone followed by a solution of potassium tert-butoxide in tetrahydrofuran (1.0 M, 0.125 mL, 0.125 mmol, 5 eq.). The resulting mixture was heated at 80° C. for 2 hours. After cooling at 23° C., the mixture was quenched by the addition of aqueous solution of NH₄Cl (1.0 M, 0.3 mL) and purified by HPLC (PRIMESPHERE C18-HC, 21.2 mm×100 mm column); mobile phase: A 10/90 CH₃CN/H₂0+5 mMol NH₄OAc, B 90/10 CH₃CN/H₂0+5 mMol NH₄OAc; gradient: 40% to 0% of A over 5 minutes; detector: UV, 220 nM; Flow rate: 20.0 mL/min. Purity of each sample was analyzed by LCMS: HPLC (Primesphere C18-HC, 4.6 mm×30 mm); mobile phase: A 10/90 CH₃CN/H₂0+5 mMol NH₄OAc, B 90/10 CH₃CN/H₂0+5 mMol NH₄OAc; gradient: 100% to 0% of A over 3 minutes; detector: UV, 250 nM; Flow rate: 4.0 ml/min.

| Ex- | | |
|---|---|---|
| am- | | Mass |
| ple | | Spectrum |
| No. | Chemical Name | m/e |
| 56 | 2-[(4-Chlorophenyl)methylthio]-4-(pyrrolidin-1- | 507 (MH⁺) |
| | yl)-N-[[4-(trifluoromethyl)phenyl]- | |
| | methyl]pyrimidine-5-carboxamide | |
| 57 | 2-[(Furan-2-yl)methylthio]-4-(pyrrolidin-1-yl)-N- | 463 (MH⁺) |
| | [[4-(trifluoromethyl)phenyl]methyl]pyrimidine-5- | |
| | carboxamide | |
| 58 | 2-[(4-Nitrophenyl)thio]-4-(pyrrolidin-1-yl)-N-[[4- | 504 (MH⁺) |
| | (trifluoromethyl)phenyl]methyl]pyrimidine-5- | |
| | carboxamide | |
| 59 | 2-[(3-Methylphenyl)methylthio]-4-(pyrrolidin-1- | 487 (MH⁺) |
| | yl)-N-[[4-(trifluoromethyl)phenyl]methyl]- | |
| | pyrimidine-5-carboxamide | |
| 60 | 2-[(2-Chlorophenyl)thio]-4-(pyrrolidin-1-yl)-N-[[4- | 493 (MH⁺) |
| | (trifluoromethyl)phenyl]methyl]pyrimidine-5- | |
| | carboxamide | |
| 61 | 2-(Cyclopropylthio)-4-(pyrrolidin-1-yl)-N-[[4- | 451 (MH⁺) |
| | (trifluoromethyl)phenyl]methyl]pyrimidine-5- | |
| | carboxamide | |
| 62 | 2-[(2-Thienyl)thio]-4-(pyrrolidin-1-yl)-N-[[4- | 465 (MH⁺) |
| | (trifluoromethyl)phenyl]methyl]pyrimidine-5- | |
| | carboxamide | |
| 63 | 2-[(2-Methyl-1-propyl)thio]-4-(pyrrolidin-1-yl)-N- | 439 (MH⁺) |
| | [[4-(trifluoromethyl)phenyl]methyl]pyrimidine-5- | |
| | carboxamide | |
| 64 | 2-(Phenylmethylthio)-4-(pyrrolidin-1-yl)-N-[[4- | 473 (MH⁺) |
| | (trifluoromethyl)phenyl]methyl]pyrimidine-5- | |
| | carboxamide | |

### EXAMPLES 65-67

Examples 65-67 were prepared by the general procedure described previously for Examples 2-18.

| Ex- | | |
|---|---|---|
| am- | | Mass |
| ple | | Spectrum |
| No. | Chemical Name | m/e |
| 65 | 4-(Trifluoromethyl)-2-(diethylamino)-N-[(5- | 357 (MH⁺) |
| | methylfuran-2-yl)methyl]pyrimidine-5- | |
| | carboxamide | |
| 66 | 4-(Trifluoromethyl)-2-(pyrrolidin-1-yl)-N-[(furan- | 341 (MH⁺) |
| | 2-yl)methyl]pyrimidine-5-carboxamide | |
| 67 | 4-(Trifluoromethyl)-2-(morpholin-4-yl)-N-[(5- | 371 (MH⁺) |
| | methylfuran-2-yl)methyl]pyrimidine-5- | |
| | carboxamide | |

We claim:

1. A method for the treatment of disorders responsive to opening of the KCNQ potassium channels in a mammal in need thereof, which comprises administering to said mammal a therapeutically effective amount of a compound of Formula I 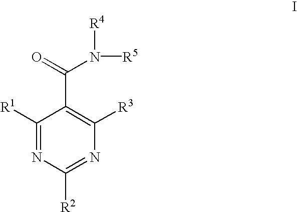
wherein
R¹ is selected from hydrogen, halogen, C₁₋₈alkyl, phenyl, phenylalkyl, C₃₋₆heterocyclic, C₃₋₆heterocyclicmethyl, —CN, —OR, —NRR, —NRNCOR or —CF₃;
R² is selected from halogen, C₁₋₈alkyl, C³⁻⁷cycloalkyl, phenyl, phenylalkyl, C₃₋₆heterocyclic, C₃₋₆heterocyclicmethyl, —CN, —OR, —NRR, —NRNCOR or —S—R;
R³ is selected from hydrogen, halogen or C₁₋₈alkyl;
R⁴ is selected from hydrogen, —CH₃ or —CH₂C₆H₅;
R⁵ is selected from hydrogen, C₁₋₈alkyl, C₃₋₇cycloalkyl, phenyl, phenylalkyl, C₃₋₆heterocyclic or C₃₋₆heterocyclicmethyl;
wherein each occurrence of R is independently selected from the group consisting of C₁₋₈alkyl, C₃₋₇alkynyl, phenyl, phenylalkyl, C₃₋₆heterocyclic and C₃₋₆heterocyclicmethyl.

2. The method of claim 1 wherein the compound of Formula I is selected from a compound having the structure 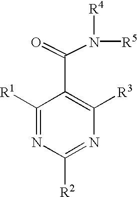
wherein
R¹ is hydrogen;
R² is selected from the group consisting of NR⁶R⁷, SR⁸, OR⁹, phenyl, and thienyl; in which said phenyl is optionally substituted with one or two C₁₋₃alkoxy groups;
R³ is selected from the group consisting of C₁₋₆alkyl, trifluoromethyl, C₃₋₇cycloalkyl, C₃₋₇cycloalkylmethyl, phenyl, amino, di(C₁₋₃alkyl)amino and pyrrolidinyl; in which said phenyl is optionally substituted with a halogen;
R⁴ is selected from the group consisting of phenylmethyl, furanylmethyl, and C₃₋₇cycloalkylmethyl; in which the phenyl of said phenylmethyl is optionally substituted with one substituent selected from the group consisting of halogen, C₁₋₃alkyl, di(C₁₋₃alkyl)amino, trifluoromethyl, trifluoromethoxy, and trifluoromethylthio; and in which the furanyl of said furanylmethyl is optionally substituted with a C₁₋₃alkyl group;
R⁵ is hydrogen;
R⁶ and R⁷ are each independently selected from the group consisting of hydrogen, C₁₋₆alkyl, C₃₋₇cycloalkyl, C₃₋₇alkynyl, phenyl, and phenylmethyl; in which said C₁₋₆alkyl is optionally substituted with a hydroxy group and in which said phenyl is optionally substituted with one or two substituents selected from the group consisting of halogen, trifluoromethoxy, and nitro; or R⁶ and R⁷ taken together with the nitrogen to which they are attached form a heterocyclic ring selected from the group consisting of pyrrolidinyl, morpholinyl, piperidinyl, homopiperidinyl, methylpiperidinyl, and 1,2,3,4-tetrahydoisoquinolinyl;
R⁸ is selected from the group consisting of C₁₋₆alkyl, C₃₋₇cycloalkyl, phenyl, phenylmethyl, furanylmethyl, and thienyl; in which said phenyl is optionally substituted with one halogen or nitro group; and wherein the phenyl of said phenylmethyl is optionally substituted with one halogen or C₁₋₃alkyl group; and
R⁹ is selected from the group consisting of C₃₋₇alkynyl, phenyl, 1-(4-fluorophenyl)ethyl, and thienylmethyl; in which said phenyl is optionally substituted with a halogen or C₁₋₃alkoxy group.

3. The method of claim 1 wherein said disorder is migraine or migraine-like attack.

4. The method of claim 2 wherein said disorder is migraine or migraine-like attack.

5. A pharmaceutical composition for the treatment of disorders responsive to opening of KCNQ potassium channels comprising a therapeutically effective amount of the compound of claim 1 in association with a pharmaceutically acceptable carrier, adjuvant or diluent.

6. A pharmaceutical composition for the treatment of disorders responsive to opening of KCNQ potassium channels comprising a therapeutically effective amount of the compound of claim 2 in association with a pharmaceutically acceptable carrier, adjuvant or diluent.
